(19) United States
(12) Patent Application Publication
(10) Pub. No.: US 2025/0333366 A1
(43) Pub. Date: Oct. 30, 2025

(54) SYSTEM AND METHOD FOR ATMOSPHERIC PRESSURE METHANE ENRICHMENT

(71) Applicant: Circularity Fuels, Inc., Redwood City, CA (US)

(72) Inventors: Stephen E. Beaton, Redwood City, CA (US); Gaurav Kamat, Redwood City, CA (US); Ford McClure, Redwood City, CA (US); Anna Lee Tonkovich, Redwood City, CA (US)

(73) Assignee: Circularity Fuels, Inc., Redwood City, CA (US)

(21) Appl. No.: 19/194,670

(22) Filed: Apr. 30, 2025

Related U.S. Application Data
(60) Provisional application No. 63/640,547, filed on Apr. 30, 2024.

(51) Int. Cl. C07C 1/12; B01J 20/04; B01J 21/04; B01J 23/28; B01J 23/46; B01J 23/755; B01J 35/00; B01J 35/57; B01J 37/02; C01C 1/04

(52) U.S. Cl. CPC C07C 1/12; B01J 20/043; B01J 21/04; B01J 23/28; B01J 23/462; B01J 23/755; B01J 35/19; B01J 35/57; B01J 37/0244; C01C 1/04

(57) ABSTRACT

The disclosed technology encompasses novel methods, systems, and materials for the efficient recycling and upgrading of carbon dioxide containing streams and their conversion into high purity methane and ultra-high purity methane. In various embodiments, the methods comprise utilizing a reactor equipped with both a sorbent and catalyst; supplementing the incoming stream with appropriate amounts of hydrogen to fully convert the carbon dioxide; initiating flow of the incoming stream in the sorbent-enhanced catalysis reactor; removing contaminants like nitrogen with sorption or reactors; drying the gas; and finally injecting the gas into the infrastructure of industrial process for the gas. Notably, the reaction of carbon dioxide with hydrogen gas to yield methane takes place at a temperature substantially consistent with the desorption stage on the catalytic unit. This invention represents a significant advancement in carbon upgrading processes, offering a highly efficient and reliable method for producing high purity methane and ultra-high purity methane.

### CROSS-REFERENCE TO RELATED APPLICATIONS

This application claims the benefit of U.S. Provisional Application No. 63/640,547 filed 30 Apr. 2024, which is incorporated in its entirety by this reference.

### TECHNICAL FIELD

This invention relates generally to the carbon deposition field, and more specifically to a new and useful system and method in the carbon deposition field.

### BACKGROUND

The invention pertains to the field of generating high-purity and ultra-high-purity methane from streams containing methane and/or contaminant carbon and nitrogen species. Specifically, it involves the use of sorbent-enhanced catalysts to convert various carbonaceous species in a stream enriched with hydrogen into high-purity and ultra high-purity methane. In certain embodiments, this process could be used to convert waste streams containing methane, carbon dioxide, and other contaminants into a high-purity methane suitable for transportation in natural gas infrastructure. In certain embodiments, this process could be used to convert a primarily hydrogen-containing stream with methane and carbon impurities into an ultra-high purity methane that could be used in industrial applications requiring high purity methane such as chemical vapor deposition (CVD), atomic layer deposition (ALD), and semiconductor manufacturing.

### BRIEF DESCRIPTION OF THE FIGURES

FIG. 1 shows graphical results from a worked example of methanation over a methanation catalyst (Ru or Ni) deposited on a porous alumina coated ceramic monolith. In this specific example, the weight loading of Ru/Al₂O₃ was 1.5 wt % with a loading of 91.5 kg/m³ as coated on a monolith of 8-cm diameter made from a cordierite dense honeycomb monolith with an open flow channel gap of about 0.9 mm for an estimated 880 parallel channels per monolith. In this specific example, the coating was achieved by dip coating with a slurry of 35 wt %-Al₂O₃ composition and 1 to 3 coatings with intervening drying to 120° C. Then, in this specific example, the alumina coated monolith was immersed in an aqueous solution of 1.5 wt % ruthenium nitrosyl nitrate followed by reduction at 300° C. for 5 hours in hydrogen at low flow rate of 2 CCM. In this specific example, the catalyst-coated monolith inlet temperature was about 210° C., flow rate is 60 SCCM, incoming gas was a 1:5 carbon dioxide to hydrogen mixture. In this graph, the horizontal x-axis represents time in seconds since the initiation of the experiment and the vertical y-axes represent the concentrations in percent of CO₂, CH₄, and CO at the outlet of the catalyst-only reactor. Before 4500 seconds, only hydrogen was flowing through the reactor. The carbon dioxide was added to the mixture of hydrogen around 4500 seconds. Thus, by the end of the experiment as shown on the graph, the reactor had reached near-steady state conditions. The decrease in methane formation and increase in carbon monoxide formation over the length of the experiment could have resulted from uneven heating, hot spot formation from the highly exothermic reaction, increasing carbon monoxide poisoning of the catalyst, and/or could have been the result of other sources of contamination.

FIG. 2 shows graphical results from one worked example of methanation over a sorbent-enhanced methanation catalyst (e.g., Na₂O assisted Ru or Ni). In this specific example, the tests were performed with a weight loading of Ru/Al₂O₃ was 1.1 wt % with a loading of 95 kg/m³ and a weight loading of Na₂O of 9.3 kg/m³ as coated on a monolith of 8-cm diameter made from an α-alumina dense honeycomb monolith with an open flow channel gap of about 0.9 mm for an estimated 880 parallel channels per alumina coated ceramic monolith. In this specific example, the coating was achieved using dip coating where the catalyst was coated first using a slurry of 35 wt % Al₂O₃ composition and 3 dip coatings with intervening dryings to 120° C. Then, in this specific example, the alumina coated monolith is immersed in an aqueous solution of 1.5 wt % ruthenium nitrosyl nitrate followed by reduction at 300° C. for 5 hours in hydrogen at low flow rate of 2 CCM. In this specific example, the sorbent was coated over the dried catalyst coating using a slurry of 6 wt % Na₂CO₃ composition with 3 dip coatings in series each with intervening drying steps to 120° C. In this specific example, the monolith temperature is about 210° C., flow rate was 60 SCCM, incoming gas was a 1:5 carbon dioxide to hydrogen mixture to repeat the same conditions as the preceding experiment (e.g., as shown in FIG. 1) with catalyst only of equal weight loading. Similar (or better) results are expected for other reaction conditions (e.g., different catalysts, different temperatures, using a plurality of thermal reactors, etc.). In this graph, the horizontal x-axis represents time in seconds since the initiation of the experiment and the vertical Y-axis represents the concentrations of CO₂, CH₄, and CO at the outlet of the sorbent-enhanced catalyst reactor. Before 1500 seconds, only hydrogen was flowing through the reactor. The carbon dioxide was added to the mixture of hydrogen around ˜1500 seconds. Thus, by the end of the graph, the reactor had reached near steady state conditions. The linear methane formation in near steady state is an artifact of the analytical method (Gas Card NG) that can only detect up to 39% methane, but the essentially complete conversion of carbon dioxide where the outlet composition was less than 50 ppm throughout the experiment, lack of carbon monoxide formation, and very high selectivity towards methane are surprising given all other variables (reactor size, input, furnace temperature, and catalyst) remain the same as FIG. 1.

FIG. 3 is a schematic representation of an example of a catalyst alone processing a feed stream that includes carbon oxides, methane, hydrogen, and H₂O. In this figure, the catalyst has a high conversion rate but low affinity for the reactant causing only partial conversion, so that this process may provide results similar to those in FIG. 1.

FIG. 4 is a schematic representation of an example of a sorbent-enhanced catalyst system. In this figure, the exemplary catalyst has a high conversion rate but low affinity for the reactant which would result in partial conversion of the carbon oxide to methane (without the presence of the sorbent that cannot catalyze carbon dioxide conversion but has a high enough affinity so that the carbon dioxide remains proximate to the catalyst). This process may provide results similar to those in FIG. 2. It should be understood from this disclosure that the active coating materials represented in this figure are one example of the present invention, and other active catalyst materials and adsorbent materials and combinations thereof could be used.

FIG. 5A is a process flow diagram of an example of the process. FIG. 5B is a table listing the reference numbers and names/descriptions of corresponding processes or fluid streams of FIG. 5A, and exemplary approximate compositions/concentrations of said fluid streams.

FIG. 6A is an exemplary process flow diagram of an example of the process. FIG. 6B is a table listing the reference numbers and names/descriptions of corresponding processes or fluid streams of FIG. 6A, and exemplary approximate compositions/concentrations of said fluid streams.

FIG. 7 is a schematic illustration comparing an exemplary sorbent-enhanced catalyst reactor surface (upper portion) to an exemplary catalyst-only reactor surface (lower portion) showing that conversion to methane may be substantially enhanced by using the sorbent-enhanced catalyst.

FIG. 8 is a schematic representation of an example of the process.

FIG. 9 is a flow chart representation of one or more exemplary steps that can be included in upgrading a waste methane stream. Note that variants of upgrading the waste methane stream can include any or all of the process steps shown in this figure in any suitable order.

FIG. 10 is a schematic representation of an example of the process integrated with a diamond growth chamber. In this example, the CVD growth substrate can optionally be cooled using carbon dioxide, where said carbon dioxide can optionally be introduced into the waste stream upcycling (e.g., to increase a reactor pressure, to shift a reaction equilibrium such as for pre-reforming, etc.).

### DETAILED DESCRIPTION

The following description of the embodiments of the invention is not intended to limit the invention to these embodiments, but rather to enable any person skilled in the art to make and use this invention.

### 1. Overview

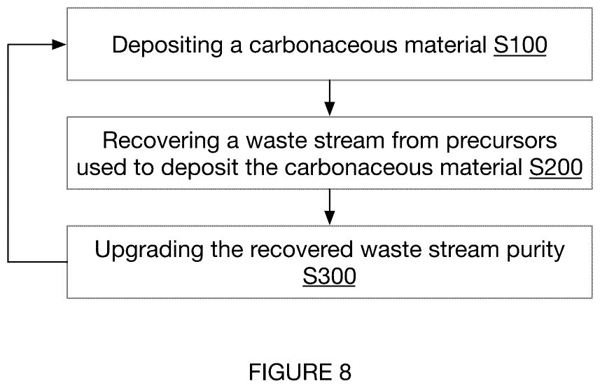

As shown for example in FIG. 8, a process can include: depositing a carbonaceous material from deposition precursors S100, recovering unused deposition precursors S200, upgrading the recovered unused deposition precursors S300, and/or other suitable processes.

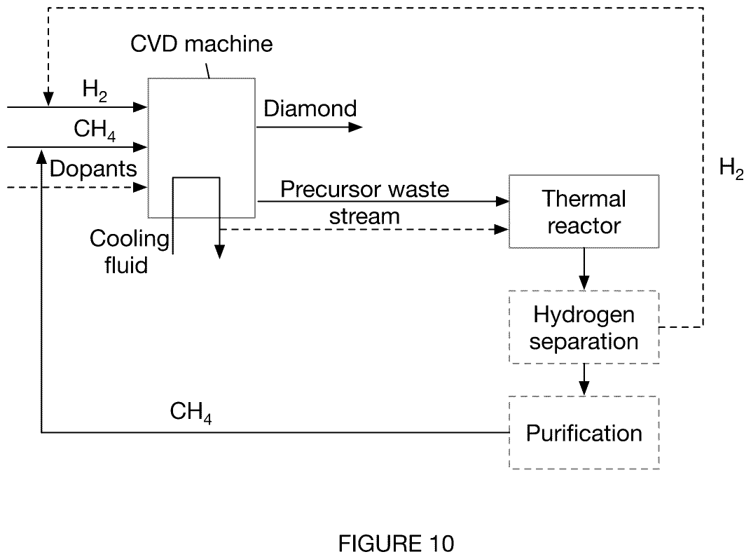

As shown for example in FIG. 10, a system can include a deposition chamber, one or more thermal reactor, one or more purifier, and/or other suitable components.

Variants of the system and/or process can function to grow a carbonaceous material and/or to upgrade waste precursor material left after growing the carbonaceous material. For instance, the system and/or process can be used with diamond growth chamber to upgrade waste from the diamond growth chamber back into methane with sufficiently high purity to enable recycle of the waste stream. However, other impure methane stream can be upgraded using variants of the process (e.g., to enable their use for carbonaceous material deposition, for high purity fuel uses, to facilitate isotopic enrichment, etc.).

### 2. Technical Advantages

Variants of the technology can confer one or more advantages over conventional technologies.

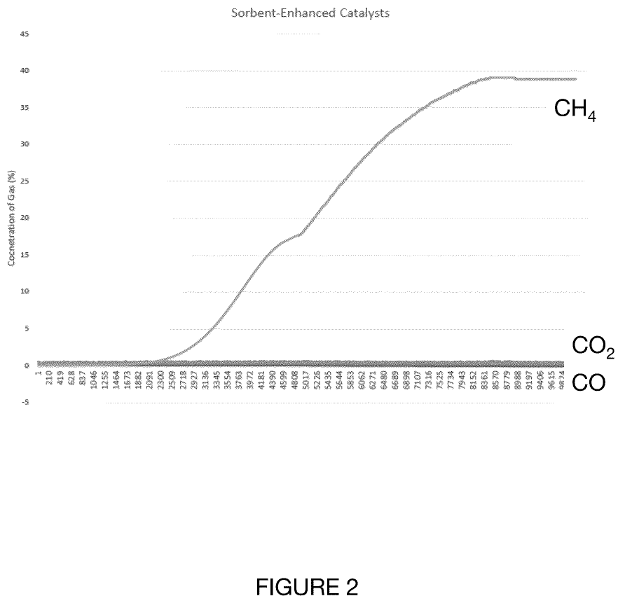

First, variants of the technology can enable continuous operation of methane waste stream upcycling. To contrast, batch mode operation for upgrading waste methane streams typically involves capturing carbon dioxide from a dilute stream and heating the system with hydrogen to convert the adsorbed carbon dioxide into methane before returning to capture mode. The inventors have discovered that benefits of this system for continuous methanation particularly focusing on non-ideal streams that contain additional reactants (e.g., reactive nitrogen species, non-methane hydrocarbons, oxygenates, dopants such as boron or phosphorous species, etc.) in addition to CO₂ and H₂. Additionally, the inclusion of a sorbent in steady-state methanation is counterintuitive—because desorption from the sorbent is significantly slower than catalytic conversion the average practitioner in this field would posit that the addition of the sorbent would decrease catalytic activity. By contrast, the inventors found that conversion by sorbent-enhanced catalysts can be performed in a continuous process, as supported by FIG. 2, yielding benefits of continuous reactor operation compared to the complexities and drawbacks of batch processing.

Second, the inventors have found the variants of the technology can result in selective production of methane from carbon oxides, particularly but not exclusively during continuous operation at near stochiometric ratios. This result was not expected as methanation is an exothermic reaction and hot spot formation leading to CO formation and coking of the reactor is common in methanation reactors. Without being limited to a single theory, having a sorbent that is capable of the endothermic release of CO₂ is believed to help balance the local temperature to prevent or minimize CO formation during sustained methanation. Additionally or alternatively, because the catalyst is covered by or in intimate contact with a layer of sorbent can prevent or reduce poison access to the catalyst and could sterically hinder formation of poisoned catalyst particles, the sorbent-enhanced catalysis system is not vulnerable to the same level of runaway CO poisoning and coke formation. Moreover, coke formation becomes more kinetically favorable at higher temperatures and variants of the technology are preferably performed at temperature below the coking temperature (e.g., at temperatures less than about 500° C.) by integrating an endothermic CO₂ desorption with the exothermic methane formation reaction, thereby controlling and/or lowering the local reaction temperature and supporting higher conversion and reduced propensity for coke formation.

Third, variants of the technology can reduce the carbon footprint of carbonaceous material deposition. Traditionally, carbonaceous material deposition vents materials from the waste stream (as being too contaminated, low purity, etc. for further use). In contrast, variants of the technology can upgrade the waste stream to return to sufficiently high purity as to enable recycling of the waste stream as deposition precursor and ultimately reducing the amount of vented material. In one specific example, when growing highly isotopically pure carbonaceous material (e.g., growing a carbonaceous material with isotopic purity greater than 99.5%, 99.9%, 99.95%, 99.99%, 99.995%, 99.999%, 99.9999%, 99.99999%, 99.999999%, 99.9999999%, etc.), the process can significantly reduce the amount of wasted precursor (which can be expensive with a cost that depends on the isotopic purity of the precursor).

Fourth, contrary to traditional chemical vapor deposition processes which use open loop operation or recirculate precursors within the chamber, variants of the method can enable closed loop operation of a chemical vapor deposition process. For example, by upgrading the waste methane, the upgraded methane can be recirculated into the chemical vapor deposition chamber, thereby increasing a carbon utilization (in some variants as high as 90% or potentially greater of carbon atoms within the waste methane stream can be reintroduced within the chemical vapor deposition chamber) and/or decreasing a carbon intensity of the chemical vapor deposition process.

However, further advantages can be provided by the system and method disclosed herein.

### 3. Process

As shown for example in FIG. 8, a process 10 can include: depositing a carbonaceous material from deposition precursors S100, recovering unused deposition precursors S200, upgrading the recovered unused deposition precursors S300, and/or other suitable processes.

The process is preferably performed continuously. However, the process and/or substeps thereof can be performed in batches (e.g., on start-up or shut-down waiting for sufficient species build up before operating) and/or with other suitable timing (e.g., in some variants one or more processes can be bypassed).

Depositing a carbonaceous material from deposition precursors S100 functions to produce a carbonaceous material from one or more precursors. S100 preferably include chemical vapor deposition (e.g., laser chemical vapor deposition, photo-initiated chemical vapor deposition, metalorganic chemical vapor deposition, hot filament chemical vapor deposition, combustion chemical vapor deposition, atomic-layer chemical vapor deposition, microwave plasma-assisted chemical vapor deposition, plasma-enhanced chemical vapor deposition, remote plasma-enhanced chemical vapor deposition, etc.). However, S100 can additionally or alternatively include physical vapor deposition, hybrid chemical-physical vapor deposition, and/or other processes.

Examples of carbonaceous materials can include diamond, doped diamond (e.g., n-type doped diamond, p-type doped diamond, degenerately doped diamond, etc.), graphene, carbon nanotubes, whisker carbon, polymeric carbon, pyrolytic carbon, amorphous carbon, fullerenes, glassy carbon, and/or other suitable carbonaceous materials. In some variants (e.g., to have highly controlled thermal and/or electrical transport properties), the carbonaceous materials can have a high isotopic purity (e.g., requiring precursor material with high isotopic purity that roughly matches that of the target isotopic purity of the deposited carbonaceous material).

Examples of precursors for deposition of carbonaceous materials can include: carbon sources (e.g., methane, ethane, ethene, ethyne, propane, butane, isobutane, pentane, isopentane, neopentane, etc.), plasma-forming molecules (e.g., hydrogen, oxygen, nitrogen, ozone, argon, neon, water, etc.), dopant precursors (e.g., gaseous or volatilizable species for introducing one or more dopant such as dopants described above), and/or other suitable species. Typically, different precursors are introduced via different ports (e.g., at different locations, angles, etc. within the chamber such as relative to the substrate whereon growth of the carbonaceous materials occurs). However, a plurality of precursors could be introduced (in some variants) from a shared port.

The precursors are preferably high purity (e.g., have a purity greater than 90%, 95%, 97%, 99%, 99.5%, 99.9%, 99.95%, 99.999%, 99.9995%, 99.9999%, 99.99999%, etc. where the percent can refer to mass, volume, stoichiometry, etc.).

Typically, a relatively small amount of precursor is actually consumed in the deposition (e.g., 2-20% of incident carbon atoms, and sometimes less, are incorporated in the deposited material) with the remainder being removed from the deposition chamber in a waste stream.

Within the deposition chamber, a variety of surfaces (e.g., the substrate, platens holding the substrate, chamber walls, etc.) can be cooled. For example, the growth substrate (e.g., diamond substrate, iridium substrate, iridium substrate impregnated with diamond, sapphire substrate, iridium-coated sapphire substrate, etc.) is preferably cooled. Often, inert gases (e.g., noble gases) are used for this cooling. However, other cooling fluids can be additionally or alternatively used. In one such example, carbon dioxide can be used as the cooling fluid (e.g., for the substrate, platens, chamber walls, etc.), where the carbon dioxide used for cooling can optionally supplement the waste stream (e.g., to facilitate dry reforming reactions, to increase the amount of methane that can be formed via methanation, etc.).

In some variants, S100 can include performing an etching operation and/or other process (e.g., scraping) for the removal of carbon soot that forms or is deposited on the chamber walls (e.g., deposited as amorphous carbon or other variants of carbon that are not desired or the target deposited carbon allotrope). As a specific example, an oxygen and/or hydrogen plasma can be used to convert the soot (or other deposited carbon) into hydrocarbons and/or carbon oxides (which can then be introduced in the waste methane stream of S200 or S300). As a second specific example, the soot (or other deposited carbon) can be oxidized (e.g., within an air, oxygen, or other oxidizing environment) to from carbon oxides and/or oxygenates (that can then be included within the waste stream of S200 or S300). When flakes, scrapes, powders, or other solid masses of carbon (e.g., soot, amorphous carbon, etc.) are removed from deposition chamber surfaces, the solid mass of carbon can be etched and/or processed (e.g., using the preceding examples or other processes for converting the carbon to oxygenates, carbon oxides, hydrocarbons, etc.) such that the resulting materials can be introduced into the waste stream (e.g., in S200 or S300).

Receiving a waste methane stream S200 functions to capture the outlet fluid stream from S100 and/or receive other streams with impure (e.g., less than about 90%) methane. The precursors from S100 are generally fully intermixed upon receipt, thereby resulting in a low methane purity stream that is no longer suitable for carbonaceous material deposition directly. Other examples of methane streams that can be upgraded to higher purity methane streams include (but are not limited to): biogas, landfill gas, digester gas (e.g., dairy farm digester gas), wastewater treatment gas, gasified biomass (e.g., grain husk, sawdust, straw, etc.), and point source capture (e.g., captured CO₂, captured CH₄, etc.), and combinations of these different methane sources. However, the streams can be partially mixed (e.g., not fully homogeneous). The streams preferably remain in fluid (e.g., gas, liquid) phase to be directly passed into S300.

In some variants, the waste stream can be processed prior to S300. Examples of processing steps can include enriching the waste stream (e.g., with water, carbon dioxide, carbon monoxide, hydrogen, other methane or hydrocarbon streams, etc.), separating components (e.g., reducing a hydrogen concentration of the waste stream where the separated hydrogen can optionally be reused in S100; sorbing SOₓ, NOₓ, phosphorous-compounds, boron-compounds, dopants, etc. from the stream to mitigate a risk of downstream catalyst poisoning; venting or purging inert fluids such as helium, nitrogen, argon, neon, krypton, xenon, etc. from the waste stream to mitigate build-up of the inert materials resulting from continued recycling; sorbing non-methane hydrocarbons, particularly but not exclusively aromatic hydrocarbons or derivatized heteroaromatic organic molecules; etc.), preheating and/or precooling the waste stream to a target temperature, pressurizing (e.g., compressing) or depressurizing the waste stream (e.g., to a pressure between about 1-20 psig), filtering the waste stream (e.g., to remove solids from the waste stream), and/or other suitable preprocessing steps.

The recovered waste stream can include one or more of: hydrogen, methane, non-methane hydrocarbons (e.g., saturated hydrocarbons, unsaturated hydrocarbons, ethane, ethene, ethyne, propane, propene, propyne, n-butane, i-butane, methylbutane, n-pentane, 2,2-dimethylpropane, toluene, benzene, etc.), nitrogen (e.g., N₂), reactive nitrogen compounds (e.g., organic nitrogen compounds such as amines, amides, imides, nitriles, imines, azides, azo compounds, cyanates, isocyanates, nitrates, nitriles, isonitriles, nitrites, nitro compounds, oximes, etc.; inorganic nitrogen compounds such as cyanic acid, ammonia, etc.; etc.), inert gases (e.g., neon, argon, helium, krypton, xenon, etc.), water, carbon oxides or oxocarbons (e.g., carbon monoxide, carbon dioxide, carbon suboxide, etc.), dopants (e.g., boron compounds such as diborane; phosphorous compounds such as phosphine; silicon compounds such as silane; arsenic compounds such as arsine; aluminium or aluminium compounds; antimony or antimony compounds; gallium or gallium compounds; sulfur or sulfur compounds; alkali metals; alkaline earth metals; chalcogenides; etc.), and/or other suitable species. In one specific example, the waste stream can have a composition that is between 75% and 99.99% hydrogen, between 0.01% and 25% methane, between 0.1 ppm and 5000 ppm for each non-methane hydrocarbon, between 0.01 ppm and 5000 ppm nitrogen, between 0.01 ppm and 200 ppm of reactive nitrogen species, between 0.0001% and 10% each for carbon oxides, between 0.01 ppm and 5000 ppm inert gases, and between 0.001 ppb and 10 ppm of each dopant (where percentages or concentrations can refer to mass percentages, volume percentages, stoichiometric percentages, etc., where the total composition adds up to about 100% where about accounts for errors in measurement methods or other trace amounts of material). In variations of this specific example, the waste stream can have a water concentration (e.g., relative humidity) between 20% and 100%.

Prior to S300, any dopants in the waste methane stream are preferably sorbed (e.g., using a chelating agent, amine sorbent, zeolite, scrubber, etc.) as these chemical species may undergo further reactions in S300 and/or can poison either or both of the catalyst or sorbent as used in steps or substeps of S300. However, the thermal reactor can in some variants be designed to be resilient to (and potentially upgrade) one or more dopant. As a specific example of such a variant, sulfur oxides may be hydrogenated by a thermal reactor to form hydrogen sulfide, carbon sulfide, carbonyl sulfide, ammonium sulfide, and/or other suitable species for sulfur deposition.

Upgrading the recovered unused deposition precursors S300 functions to upgrade (e.g., recycle, upconvert, purify, etc.) the waste stream (e.g., from S100, S200) into an upgraded methane stream (e.g., with a purity sufficient to use the upgraded methane stream in S100 or for other similar processes like S100). S300 is preferably performed using one or more thermal reactors (e.g., as described below). However, S300 can be performed using any suitable system. The thermal reactor(s) are preferably electrified thermal reactors (e.g., generate heat via induction, resistance or Joule heating, etc.). The thermal reactors preferably include a bifunctional catalyst (e.g., a combination of sorbent and catalyst material in close physical proximity). However, other thermal reactors could be used.

S300 is preferably performed at or near atmospheric pressure (e.g., at a pressure between 1 and 20 psig). However, in some variants, S300 can be performed under elevated pressure (e.g., 5 barg, 10 barg, 15 barg, 20 barg, 25 barg, 30 barg, 40 barg, 50 barg, etc.), where elevated pressure can be used as a mechanism for modifying a reaction rate, a reaction composition, a sorbent capacity, and/or can otherwise be used.

S300 is preferably performed at a temperature less than or equal to about 350° C. (which can be beneficial as less coking, soot formation, carbon monoxide formation, etc. is generally expected at this temperature). For example, S300 or substeps thereof, can be performed at 100° C., 150° C., 200° C., 225° C., 250° C., 275° C., 300° C., 325° C., and/or values or ranges therebetween. However, in some variants, one or more substeps of S300 could be performed at elevated temperatures (e.g., to change a reaction equilibrium).

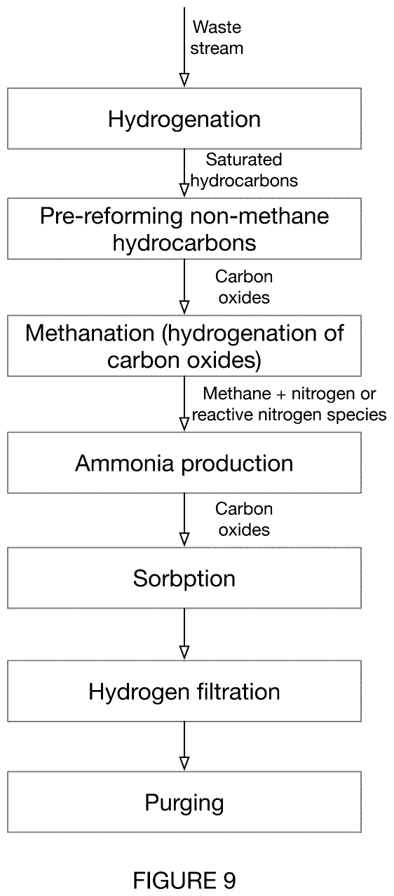

As shown for example in FIG. 9, variants of S300 can include: hydrogenation of hydrocarbons S310 (e.g., unsaturated hydrocarbons, aromatic hydrocarbons, unsaturated organic species, aromatic organic species, etc.), reforming non-methane hydrocarbons S320 (e.g., steam reforming, dry reforming, etc.), methanation of carbon oxides S330 (e.g., hydrogenation of carbon monoxide and/or carbon dioxide), ammonia production S340 (e.g., hydrogenation of nitrogen and/or reactive nitrogen species), separations (e.g., sorption S350 of ammonia, filtration of hydrogen S360, purging or venting of inerts S370, etc.), and/or other suitable steps. Typically, the reactions (e.g., hydrogenation, reforming, methanation, ammonia production) are performed in the order as written so that each down stream or subsequent process converts as much reacted material as possible (e.g., to achieve high efficiency) and/or to decrease the extent of competing chemical or physical processes (e.g., to mitigate carbon oxide saturation of sorbent for ammonia production as a specific example). However, additionally or alternatively, all of the reactions can be performed contemporaneously (e.g., be occurring at the same time), and/or can be performed in any suitable manner.

Hydrogenation of hydrocarbons S310 functions to saturate unsaturated hydrocarbons or other organic molecules from the waste steam (e.g., to facilitate downstream reforming processes, decrease soot formation or coking, etc.). Hydrocarbon hydrogenation catalysts preferably include: nickel, platinum, palladium, ruthenium, and/or rhodium that is supported on silica. The hydrocarbon hydrogenation catalyst can optionally include a promoter such as molybdenum. However, other hydrocarbon hydrogenation catalysts, support materials, and/or sorbents (or promoters) can be used. After S310, the methane stream preferably does not include a substantial amount of unsaturated hydrocarbons or other unsaturated organic species (e.g., contains less than 100 ppm of unsaturated hydrocarbons or other unsaturated organic species).

Reforming non-methane hydrocarbons S320 functions to convert hydrocarbons (particularly non-methane hydrocarbons) into carbon oxides (carbon monoxide, carbon dioxide, etc.) and hydrogen. Reforming can include steam reforming (e.g., reacting the hydrocarbons with water to produce carbon monoxide and hydrogen), direct steam reforming (e.g., reacting the hydrocarbons with water to produce carbon dioxide and hydrogen), water gas shift reaction (e.g., reacting carbon monoxide with water to produce carbon dioxide and hydrogen), and/or dry reforming (e.g., reacting hydrocarbons with carbon dioxide to produce carbon monoxide and hydrogen). Note that each of these reactions can compete and/or form an equilibrium, but generally, reaction conditions (e.g., temperature, pressure, flow rate, etc.) are such that hydrocarbons are consumed. In some variants, the hydrocarbons can include unsaturated hydrocarbons (e.g., alkenes, alkynes, benzenes or derivatives thereof, etc.) that can be directly reformed (e.g., without first being hydrogenated). Similarly, methane within the waste stream may also be reformed during this step. The pre-reforming catalyst is preferably rhodium. However, other pre-reforming catalysts, support materials, and/or sorbents (or promoters) can be used. After S320, the methane stream preferably does not include a substantial amount of hydrocarbons or other organic species (e.g., contains less than 100 ppm of hydrocarbons or other organic species). Typically, after S320, the methane stream includes at most about 2% (by mass, by volume, by stoichiometry, etc.) carbon oxides. However, higher carbon oxide concentrations (e.g., 5%, 10%, 20%, etc.) can be produced after S320 (particularly, but not exclusively, in variants that supplement or provide additional carbon dioxide for methanation).

Methanation of carbon oxides S330 functions to hydrogenate the carbon oxides (e.g., carbon monoxide and/or carbon dioxide) in the waste stream into methane. Generally, the hydrogen content of the waste stream is sufficiently high that excess hydrogen need not be added. However, additional hydrogen (e.g., generated via hydrocarbon pyrolysis where the resulting carbon is preferably oxidized into carbon oxides, water electrolysis, sulfur depolarized electrolysis, etc.) can be added (e.g., into a thermal reactor, at a particular distance along a thermal reactor, etc.) such as to achieve a target hydrogen concentration. The methanation catalyst is preferably ruthenium, rhodium, and/or nickel in intimate contact with an alkali metal oxide and/or alkaline earth metal oxide sorbent. However, other methanation catalysts, support materials, and/or sorbents (or promoters) can be used. After S330, the methane stream preferably does not include a substantial amount of carbon oxides (e.g., contains less than 100 ppm carbon oxides). In one specific example, the thermal reactor for performing methanation can have a conversion of CO₂ to CH₄ of greater than 70% (e.g., greater than 80%, greater than 90%, greater than 95%, greater than 99%, etc. per pass).

Ammonia production S340 functions to hydrogenate nitrogen and/or other reactive nitrogen species (e.g., organo-nitrogen compounds, nitrogen oxides, cyanides, etc.) into ammonia, which can be beneficial as ammonia is more readily sorbed (or otherwise separated) from the waste stream compared to other nitrogen containing species. S340 is preferably performed after S330 as nitrogen (or other reactive nitrogen species) often have a lower affinity for catalyst and/or sorbent compared to the carbon oxides (i.e., the carbon oxides typically preferably sorb and/or react on catalyst sites therefore a low concentration of carbon oxides is preferred to facilitate the nitrogen conversion reaction). The ammonia producing catalyst is preferably nickel, ruthenium, iron, and/or cobalt in intimate contact with cesium and/or barium sorbent. However, other methanation catalysts, support materials, and/or sorbents (or promoters) can be used. After S340, the methane stream preferably does not include a substantial amount of nitrogen or reactive nitrogen species (e.g., contains less than 100 ppm of nitrogen or reactive nitrogen species) as these species have been converted to ammonia.

Note that while the above reactions are described as separate, two or more reactions can be occurring contemporaneously. In one example, non-methane hydrocarbon hydrogenation and pre-reforming can occur within a first thermal reactor and carbon oxide hydrogenation (methanation) and nitrogen conversion (e.g., ammonia production) can occur within a second thermal reactor. In another example, non-methane hydrocarbon hydrogenation, pre-reforming, methanation, and nitrogen conversion can all occur within the same thermal reactor. However, any combination of the above reactions or processes can be performed in the same or distinct thermal reactors (i.e., one, two, three, or four of non-methane hydrocarbon hydrogenation, pre-reforming, methanation, and/or nitrogen conversion can be performed in each thermal reactor when a plurality of thermal reactors are used). Similarly, different reaction conditions can be achieved within the same thermal reactor (e.g., by coating different catalyst, sorbent, and/or support materials in different portions of the reactor; by having different resistivity of the substrate and thereby facilitating differential heating within different regions of the reactor; by designing different flow properties within different regions of the reactor, etc.).

Similarly, in some variants, only a subset of the above processes can be performed. As a first example of such variants (e.g., when unsaturated hydrocarbons are sorbed such as via chelation, getters, absorption, adsorption, distillation, etc. from the waste methane stream) hydrogenation of the non-methane hydrocarbons may not be performed. As a second example of such variants (e.g., when a nitrogen concentration within the waste methane stream is less than a threshold such as 200 ppm), nitrogen hydrogenation may not be performed. However, other suitable processes may be excluded in some variants of the method (e.g., because of available energy).

In some variants, the thermal reactor(s) used to perform S310, S320, S330, and/or S340 can be used to intermittently provide heat. In these variants, the reaction processes (as exothermic processes) generally produce sufficient heat to maintain the thermal reactor (at least proximal the catalyst region) at a target reaction temperature. An example of such a variant is when a total carbon oxide (inclusive of carbon dioxide and carbon monoxide) is greater than about 5% (by mass, by volume, by stoichiometry, etc.), the methanation reaction (e.g., S330) can produce sufficient heat that the thermal reactor need not introduce additional heat. However, in variations of this example with less than 5% carbon oxides (e.g., closer to 2% carbon oxides as can commonly be found in the methane stream after S320), the thermal reactor can introduce additional heat (e.g., intermittently to avoid over heating, where overheating can result in undesirable sooting and/or other problems). In some examples of these variants, the thermal reactor can intermittently provide heat (e.g., based on sensor data when a temperature of the thermal reactor or a region therein proximal a catalyst decreases below a target reaction temperature). In another variation, the thermal reactor can initiate the reactions of one or more of S310, S320, S330, and/or S340 by providing heat until the reactions are able to self-sustain the temperature of the thermal reactor. In these variants, the thermal reactor can be cooled (e.g., during times when the thermal reactor is not providing heat). For instance, an active cooling system (e.g., internal heat transfer channels with a heat transfer fluid located along between 5% and 100% of the process reaction chamber length) can be used when the thermal reactor is to be cooled. However, the process can operate without active cooling (e.g., where the exothermic heat of reaction can dissipate by heat losses).

Separation steps can function to isolate one or more materials from the methane stream (e.g., to increase a purity of the methane, to remove contaminants from the methane, etc.). Typically, separation steps are performed after S310, S320, S330, and S340. However, in some variants, separations can be performed prior to one or more of S310, S320, S330, and/or S340. In some variants, the isolated materials can be subsequently introduced into the chemical vapor deposition chamber. In other variants, the isolated materials can be used for other chemical processes (e.g., chemical synthesis, etching, in preceding steps of the method such as using separated water to increase a humidity of the waste methane stream, etc.). In yet other variants, the isolated materials can be vented or purged. Examples of separation steps include ammonia sorption S350 (e.g., using getters, chelaters, etc. such as activated carbon, zeolites, metal organic frameworks, struvite, etc.), hydrogen filtration S360, purging or venting inert gases S370 (particularly when an inert gas composition within the methane stream is greater than about 0.1% to prevent buildup of inert gases within the methane or upgraded methane stream resulting from continued recycling), desiccation of the methane stream, sorption of reactive molecules formed in preceding step(s) and/or present in a supplemental material stream (e.g., non-methane hydrocarbons, water, carbon monoxide, carbon dioxide, sulfur oxides, nitrogen oxides, reactive nitrogen species, etc.), and/or other suitable separation processes.

In a first variant of a hydrogen filtration, a fuel cell can be used to selectively oxidize hydrogen (e.g., without substantially oxidizing methane) to form water. The resulting water in the first variant can be sorbed or otherwise removed from the methane. In a second variant of hydrogen filtration, the methane stream can be pressurized (e.g., to between 100 and 800 psi) and can subsequently be passed through a membrane (e.g., a polymer membrane such as a polyimide membrane; a metallic membrane such as a platinum membrane, palladium membrane, etc.; a ceramic membrane; a carbon membrane; etc.) to separate the hydrogen from the rest of the materials in the methane stream. In the second variant, the separated hydrogen is typically high purity (e.g., ≥90% H₂ by mass, by volume, by stoichiometry, etc.) and can be reused in S100. Additionally or alternatively, the separated hydrogen can be reused in S300 (e.g., to supplement a methane stream with hydrogen, for hydrogenation reactions, etc.) and/or can otherwise be used. In some variants, S360 may not be required (e.g., a methane stream with a high concentration of hydrogen but no significant i.e., greater than about 0.1% concentration of other species can be directly used in S100).

In some variants of S300, a plurality of thermal reactors are used to perform the waste stream upgrading processes. As one illustrative example, a first thermal reactor (that can optionally exclude catalyst and/or sorbent) can be used to preheat the waste stream (e.g., to a target thermal reactor temperature) while a second thermal reactor (e.g., that includes one or more catalyst and/or sorbent) can act as a site of the chemical reactions. As a second illustrative example, separate thermal reactors can be configured for (e.g., optimized for, targeted to, etc.) each waste stream upgrading reaction (e.g., one can be configured for hydrocarbon hydrogenation, one for hydrocarbon reforming, one for methanation of carbon oxides, one for ammonia production, etc. such as by tuning one or more of a catalyst, support material, sorbent, temperature, pressure, waste stream composition or inclusion of additional material to modify the composition, etc. used in the thermal reactor). As a third illustrative example, a plurality of thermal reactors can each be operated at a different temperature (e.g., one at 150° C., one at 200° C., one at 250° C., one at 300° C., etc.). However, a plurality of thermal reactors can otherwise be used.

S300 can optionally include compressing or otherwise pressurizing the upgraded methane stream (e.g., the high purity methane stream resulting from performing one or more of the steps of S300 as described above) to facilitate introduction of the upgraded methane stream into S100 (e.g., for further deposition, for combining with a second high purity methane stream, for combining with a hydrogen stream, etc.).

After S300 (and any substeps such as S310, S320, S330, S340, S3550, S360, S370, etc. that may be performed), the methane stream is preferably high purity methane (e.g., ≥90% methane, ≥95% methane, ≥97% methane, ≥99% methane, ≥99.5% methane, ≥99.9% methane, ≥99.95% methane, ≥99.995% methane, ≥99.999% methane, ≥99.9999% methane, etc., where the percentage can refer to a mass percentage, volume percentage, stoichiometric percentage, etc.). When high purity methane is not produced, the methane stream can be recycled through S300 (e.g., until high purity methane is formed) and/or additional processing steps could be performed to improve the purity of the methane stream.

### 4. Thermal Reactor

A reactor system can function to convert a stream (also referred to as a waste stream, waste methane stream) containing hydrogen, carbon (e.g., carbon dioxide CO₂, carbon monoxide CO, methane, nonmethane hydrocarbons such as ethane, propane, butane, pentane, hexane, benzene, toluene, cyclopentane, etc.), nitrogen (e.g., N₂, HCN, NO₂, NO, or other nitrogenous species such as amines, amides, imides, nitriles, imines, azides, azo compounds, cyanates, isocyanates, nitrates, nitriles, isonitriles, nitrites, nitro compounds, oximes, etc.), inert gases, other gaseous species (e.g., phosphine, silane, borane, etc.) and convert it to an upgraded gas mixture (e.g., high-purity methane stream, a stream that includes a mixture of hydrogen and methane with less than 1% other species, etc.). As one example, an incoming stream can be composed essentially of between about 0.01%-99% methane, 0.1%-75% CO₂, 0%-5% CO, 0%-99% H₂, and 0% to 10% nitrogen species.

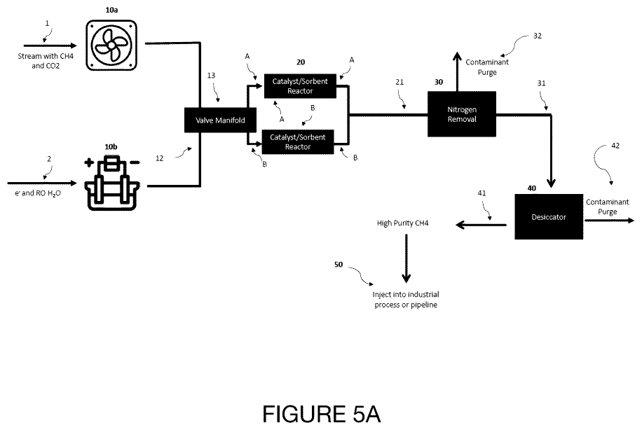

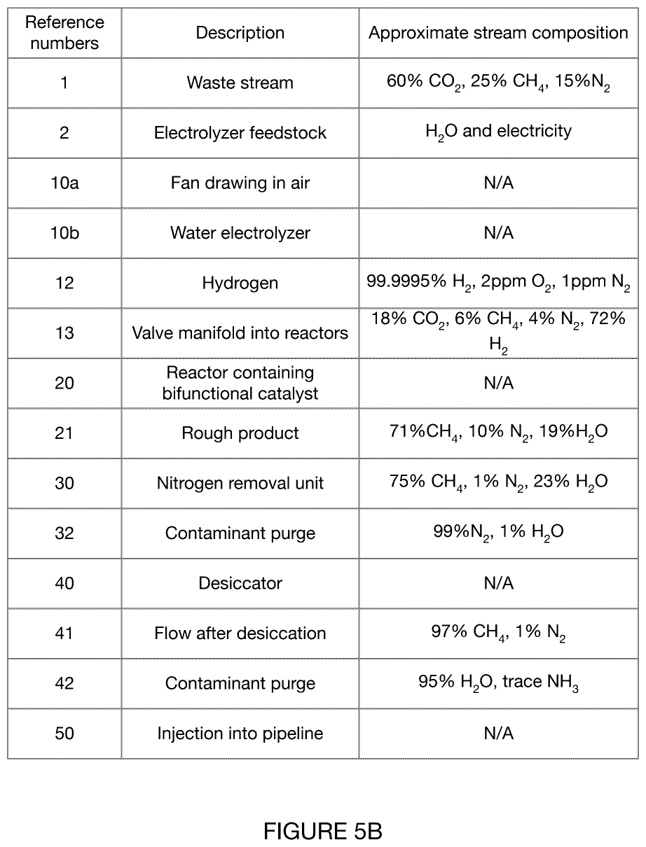

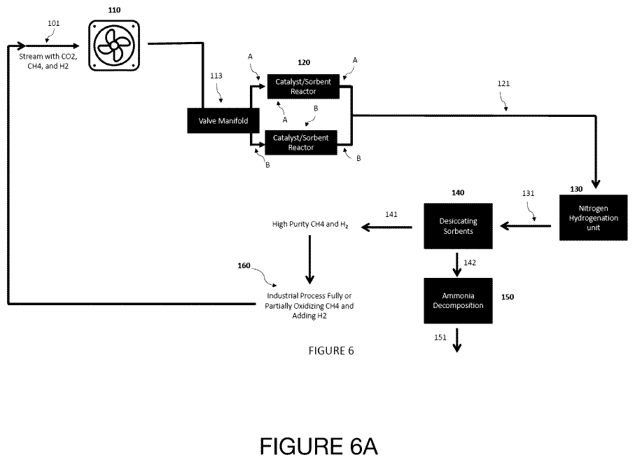

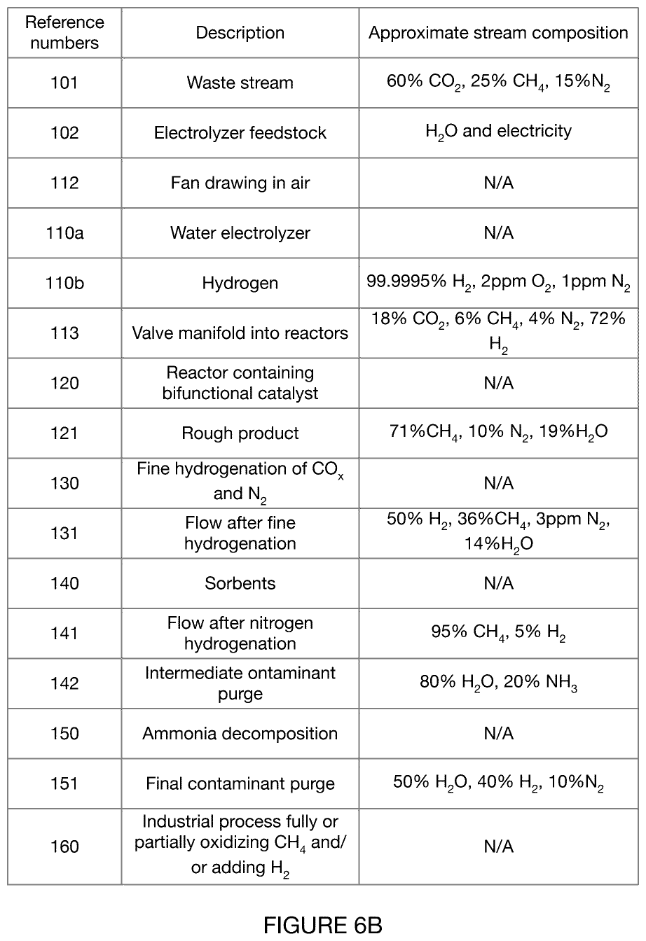

In one example (as shown for instance in FIGS. 5A and 5B), the incoming stream is from a mature landfill, with an exemplary composition of about on average 60% CO₂, 25% methane, 15% N₂, and other trace contaminants. In a second example (as shown for instance in FIGS. 6A and 6B), the incoming stream can be an exhaust stream from a CVD machine with an exemplary composition of about 95% H₂, 1% CO, 1% CO₂, 2% methane, 0.1% nitrogen species, and other trace contaminants. However, the waste stream can be from other suitable processes. The incoming waste stream can have undergone cleanup procedures (e.g., to remove problematic trace contaminants such as SiH₄, SO₂, SO₃, NO₂, NO₃, B₂H₂, B₂H₄, B₂H₆, B₄H₁₀, B₅H₉, PH₃, AsH₃, SbH₃, H₂S, H₂Se, or other species particularly, but not exclusively, those which can poison or contaminate catalysts or sorbents).

The thermal reactor preferably includes a matrix of sorbent-enhanced catalysts to substantially convert the nonmethane carbon components to methane. The sorbent-enhanced catalysts can also reduce the nitrogen concentration and other contaminants to acceptable levels.

The catalyst component (e.g., matrix of sorbent-enhanced catalysts) can include metals (such as ruthenium, nickel, platinum, rhodium, copper, cobalt, iron, osmium, palladium, iridium, rhenium, cobalt, manganese, potassium, combinations thereof, etc.), metal oxides (such as ruthenium oxide, nickel oxide, platinum oxide, rhodium oxide, copper oxide, cobalt oxide, iron oxide, osmium oxide, palladium oxide, iridium oxide, rhenium oxide, combinations thereof, etc.), a composite such as between iron, ruthenium, and/or osmium with lithium, sodium, potassium, rubidium, cesium, manganese, rhenium, etc.), and/or other suitable catalyst species.

Within the matrix of sorbent-enhanced catalysts, the catalyst is preferably in intimate contact with a sorbent component in intimate contact within the reactor. Examples of sorbent materials include alkali metals (e.g., lithium, sodium, potassium, rubidium, cesium), alkaline earth metals (e.g., beryllium, magnesium, calcium, strontium, barium), alkali metal oxides (e.g., lithium oxide, sodium oxide, potassium oxide, rubidium oxide, cesium oxide), alkaline earth metal oxides (e.g., beryllium oxide, magnesium oxide, calcium oxide, strontium oxide, barium oxide), amine functionalized materials, metalorganic frameworks (MOFs), activated carbon, zeolites, and/or other suitable sorbents. The sorbent materials preferably have a high surface area (e.g., exceeding 10 m²/g such as between 10 to 1000 m²/g, 20 to 500 m²/g, etc.). The sorbent materials can have a sorption capacity in the range of 10 to 20,000 micromoles CO₂ per gram of adsorbent, which can be beneficial for ensuring sufficient affinity for the nonmethane species in the gas stream to hydrogenate on active catalyst sites in close molecular proximity (e.g., separated by less than the thickness of the coating layer) to the sorbent site.

The catalyst and sorbent coatings are preferably coated with a total weight loading of greater than 0.7 mg/cm² (e.g., 1 mg/cm², 2 mg/cm², 5 mg/cm², 10 mg/cm², 20 mg/cm², 50 mg/cm², 100 mg/cm², values or ranges therebetween, etc.).

The ratio of catalyst to sorbent ratio can be a value between 1:100 to 1:5.

The sorbent and the catalyst are preferably disposed on a support material. Examples of support materials include (but are not limited to) microporous materials such as aluminum oxide (Al₂O₃), ceria (CeO₂), zirconia (ZrO₂), silica (SiO₂), zeolites (SiO₂—Al₂O₃), titania (Ti₂O₃), combinations thereof, and/or other suitable support materials.

The support material can be disposed on a substrate. The substrate preferably has open gap flow channels above the catalyst and sorbent layer or layers, which can result in a small pressure drop (e.g., a total system pressure loss less than about 20 psi such as 0.01 torr, 0.05 torr, 0.1 torr, 0.5 torr, 1 torr, 5 torr, 10 torr, 50 torr, 100 torr, 200 torr, 500 torr, 750 torr, 100,000 Pa, 150,000 Pa, etc.; a system loss per flow channel length of the substrate less than about 100 torr per cm such as 0.01 torr/cm, 0.05 torr/cm, 0.1 torr/cm, 0.2 torr/cm, 0.5 torr/cm, 1 torr/cm, 2 torr/cm, 5 torr/cm, 10 torr/cm, 20 torr/cm, 50 torr/cm, 110 torr/cm, etc.; etc.). However, the substrate can additionally or alternatively have a tortuous flow path (e.g., generated by a particulate or fixed bed catalyst and sorbent, a foam catalyst reactor, etc.). In one variant, the support material, sorbent, and catalyst can be layered on a ceramic honeycomb monolith, metallic honeycomb or other open flow channel monolith, mesh reactor, or another reactor.

In an alternate variant, the catalyst, support material, and/or sorbent can be coated on the walls of plates or other structures to create a flow channel that is circumscribed by the bifunctional coating. The bifunctional coating material may be placed on all sides of the flow channel such that the channel is fully or substantially 100% circumscribed by the catalyst (as might be achieved with a monolith structure that is dip coated). In an alternate variant, the combined catalyst and/or sorbent can be coated on a single plate such that the maximum catalyst and/or sorbent coverage is about 50% of the circumscribed flow channel. In some variations (e.g., to accommodate internal wall features and struts used for mechanical supports or manufacturing aids), the catalyst and/or sorbent may cover only 40% (or less) of the circumscribed flow channel.

The flow path, reaction channel and/or reaction chamber can have a polygonal cross-section (e.g., square, rectangular, triangular, trapezoidal, hexagonal, etc.), ovate cross-section (e.g., oval, elliptical, circular, etc.), and/or can have any cross-sectional shape as made possible by manufacturing methods. For at least a portion (e.g., 10%, 20%, 33%, 50%, 66%, 70%, 75%, 80%, 90%, 95%, 99%, etc.) of the total reactor length, the catalyst and/or sorbent coating preferably circumscribes at least 40% of the reaction flow channel. However, other suitable portions of the flow channel can be coated with one or more of the catalyst and/or sorbent. Within the reaction channel, the farthest orthogonal distance perpendicular to the flow direction from the reacting gas as flowing in the channel to the coating of catalyst and sorbent on the circumscribed wall is less than about 10-mm (e.g., 10 μm, 20 μm, 50 μm, 100 μm, 200 μm, 500 μm, 1 mm, 2 mm, 5 mm, etc.) for at least 50% of the reactor flow length.

The process flow can be laminar, turbulent, and/or in the transition flow regime in the reaction chamber. In one example the process flow can be in transition flow regime as defined by a Reynolds number between about 2200 and 8000. In another example, the flow can be turbulent as defined by a Reynolds number greater than about 8000. In yet another example, the process flow can be laminar with a Reynolds number less than about 2200.

In a preferred embodiment, the substrate is made of or contains a layer of a resistive material (e.g., nickel-chromium alloys, iron-chromium-aluminum alloys, copper-nickel alloys, combinations thereof, etc.), where the resistive material can be heated (e.g., for preheating, overcoming heat losses, add heat for an endothermic reaction, etc.) by applying an electrical current through the substrate. However, additionally or alternatively, resistively heated materials can be incorporated into segments of the reactor to radiatively, convectively, and/or conductively heat the entire reactor and gas stream, the thermal reactor can be heated via exothermic reactions (e.g., methanation, oxidation, hydrogenation, etc.), and/or other suitable materials can be used for the thermal reactor (e.g., cordierite, alumina, silicon carbide, etc.).

The support material, catalyst, and/or sorbent can be coated onto the substrate (and/or other layers) using dip, flow, or spray coating or other methods. In some examples, the catalyst and sorbent can be mixed and form a slurry prior to coating on the reactor walls. In another example, the catalyst and sorbent can be coated as distinct layers (e.g., where the catalyst layer can be coated closest to the wall that defines the flow channel and coated below a layer of sorbent, where the catalyst layer is coated farthest from the wall that defines the flow channel and coated on top of a layer of sorbent, forming an alternating stack of catalyst and sorbent layers, etc.). The thickness of the combined catalyst and adsorbent layer or sum of the distinct layers is typically between 5 microns and 500 microns on average (e.g., 10 μm, 20 μm, 50 μm, 100 μm, 200 μm, 250 μm, 300 μm, values or ranges therebetween, etc.).

As a specific example, the catalyst can be applied onto the support material through impregnation, ion-exchange, or precipitation. The support material, catalyst, and/or sorbent can (individually, collectively) have with a coating thickness ranging from about 1 micron to about 200 microns (e.g., 2 μm, 5 μm, 10 μm, 20 μm, 30 μm, 50 μm, 100 μm, 120 μm, 140 μm, 150 μm, 175 μm, 190 μm, 205 μm, etc.). Typically, the active catalyst loading ranges from 0.01% to 20% (e.g., about 0.1 to 10% by weight of the total composition of support material, catalyst, and sorbent). However, other catalyst loading ranges can be used (e.g., contingent upon the specific catalyst, the target reaction(s), etc.). Similarly, the sorbent loading and support material can range from 0.01% to 20% (e.g., about 0.1 to 10% by weight of the total composition of support material, catalyst, and sorbent). However, other sorbent loading ranges can be used (e.g., contingent upon the specific catalyst, the target reaction(s), etc.).

In one variant, the coating layers can form an egg-shell structure (e.g., with a first layer of sorbent with an outer layer of active catalyst). In a second variant, the sorbent and catalyst can form an egg yolk structure where the catalyst layer is interior relative to the bulk flow path and the sorbent is at the exterior surface facing the bulk flow path. In a third variant, a coating or active material can be incorporated (in addition to the sorbent-enhanced catalyst) to selectively capture and/or convert a nitrogen-containing species (e.g., to improve the purity of the resulting methane rich product stream). In a fourth variant, the catalyst and sorbent can be mixed within a slurry to coat together as a single layer on the monolith or surface.

In some variants, a combinations of the preceding embodiments can be used (e.g., such that different structures are used at different axial locations along a flow length within the reactor). These variants can, for example, promote hydrocarbon hydrogenation, prereforming, methanation of carbon oxides, and/or nitrogen species hydrogenation at different locations along the reactor.

### 4.1 Example of a Sorbent-Enhanced Catalytic Reactor in Use

When the incoming stream (e.g., waste stream, effluent gas stream) is above 350° C. (e.g., 375° C., 400° C., 500° C., etc.), the incoming stream can be cooled (e.g., to a temperature in the range between 100° C. and 350° C.). The incoming stream can be cooled, for example, by addition of a cooler stream (e.g., methane stream, H₂ stream, steam, carbon oxide stream, etc.), with a heat exchanger (e.g., using a separate heat transfer fluid, using a fin or similar structure to facilitate thermal losses such that the effluent gas stream temperature is reduced to the desired inlet temperature for the downstream conversion reactor(s)), and/or using other suitable processes (e.g., compression of the effluent waste stream at constant pressure). The waste stream can then be fed into a thermal reactor that is heated to between 150° C. and 300° C. The thermal reactor is preferably electrically heated (e.g., via Joule or resistive heating, inductive heating, etc. where the power can in some variants be dynamically adjusted based on the reaction(s) exotherm).

The effluent of the sorbent-enhanced catalyst reactor can then passed through a system of reactors or sorbent materials that can capture or convert nitrogen contaminants (e.g., nitrogen, reactive nitrogen species) from the methane stream. The system of reactors and/or sorbent materials can include a solid catalyst (such as iron, cobalt, nickel, platinum, palladium, rhodium, rhenium, ruthenium, osmium, iridium, combinations thereof, etc.); an alkaline aqueous solution of iron, potassium, or sodium; getter materials (e.g., titanium, aluminum, magnesium, barium, thorium, zirconium, cesium, etc.); or other nitrogen molecule sorbents. Alternatively, the effluent can be vented or purged (e.g., to remove inert gases such as helium, neon, argon, etc.), passed through materials that convert dilute nitrogen to other more reactive molecules such as ammonia, NOₓ, or other nitrogen molecules for subsequent capture and potential reconversion, passed through hydrogen selective filter (e.g., a polymer filter, palladium filter, etc. typically at an elevated pressure), and/or can otherwise be processed. In one example, the effluent from the methanation reaction can be pressurized to between about 1 and 20 bar and passed through a reactor containing nitrogen activation catalysts (e.g., ruthenium, nickel, iron, platinum, rhodium, copper, cobalt, group VIII transition metals, their respective oxides, combinations thereof, etc.). In another example, the effluent from the methanation reaction can be passed through immobilized nitrogenase enzymes.

The effluent of the nitrogen capture/conversion equipment can then passed through a system that removes molecules with a boiling point above −40° C. (or some other temperature greater than the boiling point of methane at the pressure of the methane stream). In one specific example, these molecules can be captured using a sorbent such as molecular sieves, zeolites, MgCl₂, CaCl₂, BaCl₂, and/or Amberlyst 15. These sorbents can then be regenerated (after they near saturation with the liquid). In a second specific example, these molecules can be removed using a condenser unit that chills the gas stream to below −40° C. (or another target temperature) where that target molecules to be separated liquify. This system can include between any suitable number of sorbents and/or condensers (e.g., one, two, five, ten, etc.) that can serve the same or different purposes (e.g., operate at different temperatures, sorb different molecules, are redundant, etc.).

The effluent of the nitrogen capture/conversion equipment and/or condensers or sorbents can be purged or vented (e.g., to remove inert gases from the methane stream).

The effluent of the nitrogen capture/conversion equipment can then be processed to separate hydrogen from the methane. For example, the hydrogen and methane stream can be pressurized and passed through a hydrogen selective membrane (e.g., polymer membrane, palladium membrane, etc.). In another example, the hydrogen and methane stream can be passed through a hydrogen electrolyzer (or other system that selectively oxidizes hydrogen) to convert the hydrogen into water (without substantially reacting the methane) followed by removal of water from the methane (e.g., via dessication, sorbtion, condensing, getting, etc.).

The products separated by the nitrogen capture/conversion sorbent or condenser units can be separated and either recycled or remediated and disposed of. In one example, these products are primarily H₂O and ammonia. The H₂O and ammonia can be separated by collecting the products at different temperatures or with different sorbent materials and/or other purification method. The H₂O can be recycled whereas the ammonia can be decomposed to N₂ and H₂ prior to venting to atmosphere (where the hydrogen can optionally also be recycled such as in the CVD deposition chamber). The ammonia decomposition can be performed using a reactor with an ammonia decomposition catalyst (e.g., ruthenium, nickel, iron, platinum, rhodium, copper, cobalt, group VIII transition metals, their respective oxides, combinations thereof, etc.). The ammonia decomposition catalyst can optionally be doped with an alkali metal (e.g., lithium, sodium, potassium, rubidium, cesium), alkaline earth metal (e.g., beryllium, magnesium, calcium, strontium, barium), and/or oxides thereof.

After the treatments (e.g., nitrogen-removal, condenser, purging, hydrogen separation, etc.), the product stream is primarily methane (e.g., high purity methane) and is preferably substantially free of H₂O, carbon contaminants (e.g., carbon oxides, non-methane hydrocarbons, etc.), nitrogen contaminants, inert gases, and/or dopants. The methane can optionally be pressurized (e.g., to between 20 and 100 bar such as for storage in a tank). The methane stream can optionally be supplemented with H₂ or methane (e.g., to change the concentration of the constituents in the gas). In variants where it occurs, this supplementation can happen either in the tank or between the tank and reuse in the reactor (e.g., CVD chamber) and can be either manually controlled or automatically controlled to create a target concentration of methane, hydrogen, or one of the contaminants.

In certain embodiments, systems (e.g., thermal reactors, condensers, hydrogen separation units, nitrogen separation units, etc.) can exist in duplicate (e.g., to ensure that one is always in operation while the other is regenerating).

Additionally, there can be one or more sensors throughout the flow path (e.g., to determine the makeup of the gas, to determine the pressure of the gas, to determine the temperature of the gas, etc.). In one embodiment, these can include infrared sensors tuned to different frequencies of the carbon and nitrogen contaminant gases and/or the infrared signature of hydrocarbons. In another embodiment, bleed streams can be fed to a mass spectrometer to determine the composition of the gas. These sensors can then be used to determine the concentration of the gas in the feed and/or product tank. A user interface can display the current concentration of gas (at each sensor location) as well as the states of the tanks.

### 4.2 Example 1

In an illustrative example of the system, the sorbent-enhanced catalysts can be embedded on a ceramic cordierite monolith. These monolith pieces can be prepared by first coating the cordierite with a layer of Al₂O₃. The Al₂O₃ layer is approximately 100 microns thick. The Al₂O₃ can then be coated in a washcoat of ruthenium, followed by a washcoat of sodium carbonate. The combined washcoat of catalyst and sodium carbonate is approximately 20 microns thick. The sorbent-enhanced catalyst loaded monoliths can be placed in a tube furnace and heated to between 200° C. and 450° C. (e.g., via resistive heating). A waste stream can be introduced to the tube furnace at near atmospheric pressure. The hydrogen is in significant excess to the stoichiometric ratio for methanation. In any ratio great than about 1:10 of carbon oxide to hydrogen (e.g., 1:15, 1:20, 1:30, 1:50, 1:100, etc.), this system can substantially convert the CO₂ to methane with only trace amounts of CO₂ (e.g., about 10 to about 50 ppm or less) remaining in the product stream.

### 4.3 Example 2

In a second illustrative example of the thermal reactor or reactive module thereof, the sorbent-enhanced catalysts can be embedded on a foil form from an iron chromium alloy foil. Such a device can be referred to as a metal monolith even though they can include more than one piece. The material can be heat treated in an oxidizing environment (e.g., air, oxygen, ozone, etc.) to form a layer of a-alumina with a thickness of about 1-micron. The surface is then coated with a porous alumina catalyst layer and supported porous adsorbent layer or layers. The Al₂O₃ can then be coated or impregnated with in a washcoat of nickel or ruthenium (and/or other catalyst materials as described above), followed by a washcoat of calcium carbonate (and/or other sorbent materials or sorbent forming materials as described above). The combined washcoat of catalyst and calcium carbonate can be between about 10 and 100 microns thick. The metal monolith can be combined with resistively heated elements internal to a tube containing the metal monolith. These resistively heated materials can be dispersed on the metal monolith and/or can be in discrete segments of the metal monolith. Thermocouple and regulation methods can be used to maintain the metal monolith between 200° C. and 450° C. and after initial sintering and reduction, a waste stream can be introduced to the tube furnace. The reaction can be exothermic such that internal heating elements can be used to preheat the reaction to initiate the process and/or to overcome heat losses (e.g., dependent on the surface area to volume ratio of the thermal reactor).

The hydrogen can be in a near stoichiometric ratio with the carbon oxides for methanation. In any ratio from about 1:4 through 1:6 of carbon dioxide to hydrogen, this example can substantially fully converts COₓ to methane with only trace amounts of COₓ remaining in the product stream (as shown for example in FIG. 2).

### 4.4 Example 3

In a third specific example (as shown for instance in FIG. 5A and FIG. 5B), CO₂ from a stream containing CH₄, CO₂, CO, and other contaminants commonly found as a byproduct of municipal waste decomposition can employ variants of the reactor system and/or method to selectively convert the carbon oxides to methane and/or selectively remove (e.g., via conversion and/or sorption processes) contaminants to recover and/or generate renewable natural gas.

In one variation of this example, the off-gas stream from a landfill can include about 60% CO₂, 25% CH₄, and 15% N₂. Trace contaminants that could be harmful to the catalytic process (e.g., reactive nitrogen species, organosilicon compounds, sulfur oxides, sulfur hydride, nitrogen oxides, etc.) could be removed (e.g., using scrubbers, sorbents, etc.). The off-gas stream can be combined with a stream of H₂ (e.g., from water electrolysis). The CO₂ in the off-gas stream can be combined with the H₂ to produce methane using a catalytic monolith and/or other structured or designed low pressure drop catalytic reactor (e.g., with a pressure drop less than 750 torr such as 5 torr, 10 torr, 50 torr, 100 torr, 500 torr, etc.). The catalyst(s) and/or sorbent(s) are preferably coated on a monolith as described in Illustrative example 1 or illustrative example 2.

The nitrogen-based compounds (e.g., nitrogen, reactive nitrogen species) can then be substantially removed through pressure swing adsorption, temperature swing adsorption, or membrane separation. In some variations of this illustrative example, the nitrogen and/or reactive nitrogen species can be hydrogenated to form ammonia (e.g., using a reactor module substantially the same as that for carbon oxide hydrogenation) which can more readily separated from the methane (e.g., via ammonia getting, ammonia sorption, etc.) and can be used for other processes (e.g., can be used as a commodity chemical). The upgraded methane gas (after substantially complete removal of the nitrogen content) can be dried (e.g., by sorbents, condensation units, etc.) and injected into a pipeline (e.g., natural gas pipeline, CVD precursor stream, etc.).

### 4.5 Example 4

In a fourth illustrative example (as shown for instance in FIG. 6A and FIG. 6B), variants of a thermal reactor and/or method can generate methane for Industrial Process Fully or Partially Oxidizing CH₄ and Adding H2 (of which chemical vapor deposition (CVD) is one example that is used in this illustrative example, but is not necessarily the only application) from the CVD process's waste streams and/or other high temperature processes (while avoiding a build-up of carbon-based compounds, nitrogen-based compounds, and/or inert gases) by combining the use of selective conversion and/or selective adsorption processes.

The off-gas stream from an exemplary CVD machine operations can include CH₄, CₓH_{y} (for x>1 and y≥2), CO₂, CO, H₂, N₂, inert gases, and/or dopants. In this illustrative example, the dopants are preferably removed prior to further processing (e.g., via sorbing getting, chelating, etc. as the dopants can poison catalysts and/or sorbents and/or can otherwise be undesirable in downstream processes). The conversion of the off-gas stream to a high purity methane stream (e.g., suitable for recycle and reuse in the CVD process) is preferably accomplished using one or more catalytic monoliths and/or other structured or designed low pressure drop catalytic reactor (e.g., with a pressure drop less than 750 torr such as 5 torr, 10 torr, 50 torr, 100 torr, 500 torr, etc.). The catalyst(s) and/or sorbent(s) are preferably coated on a monolith as described in Illustrative example 1 or illustrative example 2.

In one specific variation of the fourth illustrative example, for a feed composition of 3% by volume CO₂ in a mixture comprising hydrogen at 1 bara and 300° C., the equilibrium composition of CO₂ is substantially less than 1 ppm with the equilibrium product mole fraction of methane near 3.19%. The volume % higher than the feed is due to the reduction in the total number of moles for the Sabatier reaction. At a lower pressure of 0.01 bara or 7.5 torr the equilibrium composition of CO₂ is about 5 ppm. Thermodynamics determines the potential composition at infinite time. A practical reactor can have kinetic limitations in reducing the conversion of CO₂ to methane in a reactor of a practical volume, mass transfer limitation (e.g., due to laminar or turbulent flow within the channels), and/or can have other limitations. Therefore, the reactor likely does not achieve the results predicted based on thermodynamic equilibrium. When operated under a kinetic or mass transfer limited regime, a catalyst without an infinite rate of reaction will not fully convert to the thermodynamic potential.

In variations of this illustrative example, the CₓH_{y} species can be hydrogenated to CₓH₂ₓ₊₂ (or potentially into cyclic hydrocarbons that still include degrees of unsaturation equal to the number of cycles within the hydrocarbon). The CₓH_{y} and/or CₓH₂ₓ₊₂ species can then be reformed into CO and/or CO₂ (e.g., contemporaneously with forming H2 such as via steam reforming, dry reforming, etc.). The hydrocarbon hydrogenation reaction preferably occurs using a catalytic monolithic employing one or more of nickel, platinum, palladium, ruthenium, and/or rhodium (on a silica support) that can optionally include molybdenum (and/or other suitable promoter or sorbents such as activated carbon, zeolites, MOFs, etc.). The reforming preferably occurs using a catalytic monolithic employing a rhodium catalyst that can optionally include suitable promoters or sorbents (e.g., activated carbon, zeolites, MOFs, molybdenum, etc.).

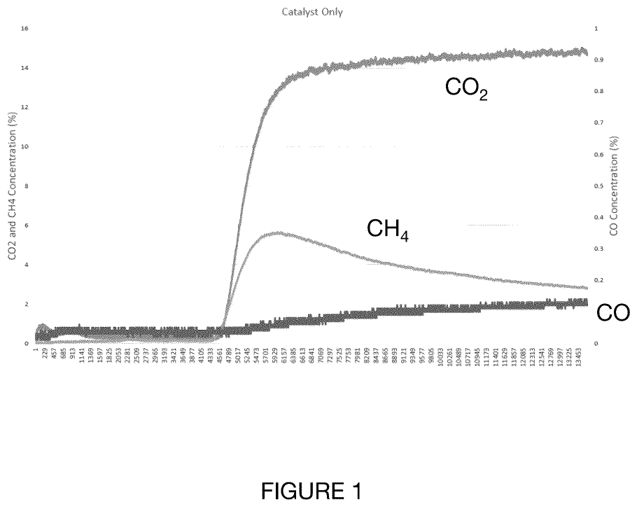

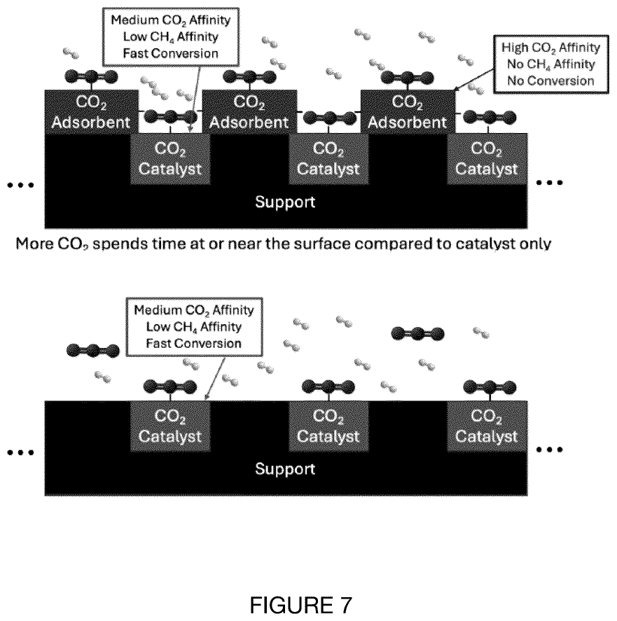

With the use of a catalyst (e.g., ruthenium, rhodium, nickel, etc.) that is not enhanced by inclusion of a sorbent, CO₂ is shown to partially convert to methane (as shown for example in FIG. 1 where only 5% of the CO₂ is converted to CH₄). In contrast, a catalyst (e.g., ruthenium, rhodium, nickel, etc.) in intimate contact with a sorbent (e.g., alkali metal oxides, alkaline earth metal oxides, etc.) can achieve conversion of CO₂ to methane that approaches the thermodynamic limit (as shown for example in FIG. 2). Without being limited to a single theory, one cause for this difference is believed to be that the sorbent integrates intimate CO₂ sorption near the active catalyst sites thereby greatly increasing reaction rate (as shown for example in FIG. 7 comparing the two situations)

Continued recycling of the methane can result in accumulation of dilute nitrogen species and/or inert species in the recycled flow process when not removed. The inert species and/or nitrogen can be removed by venting or purging, by converting the nitrogen species into a sorb-able or otherwise separable form of nitrogen (e.g., a nitrogen compound that is readily separated from methane), and/or can otherwise avoid this build up. As an example of nitrogen species separation, and preferably after substantially complete removal of carbon oxides (to avoid catalyst poisoning), the nitrogen species (e.g., nitrogen, reactive nitrogen species, nitrogen-based compounds, etc.) can be catalytically converted to ammonia. The nitrogen species can be hydrogenated (e.g., converted to ammonia and potentially methane for organic nitrogen containing species) using catalytic monolith that includes a nickel, iron, ruthenium, cobalt, or combinations thereof catalyst with an optional promoter or sorbent (such as cesium, barium, cesium oxide, barium oxide, etc.). The nitrogen species hydrogenation preferably proceeds at a temperature between 150° C. and 400° C. The resulting ammonia can be sorbed on a sorbent (e.g., zeolite, zirconia, alumina, MOF, active carbon, etc.) with an ammonia capacity between 0.001 and 1 gram ammonia per gram of sorbent. When the sorbent is nearly saturated (e.g., between 10 and 100% of full or saturation capacity), the ammonia can be desorbed (e.g., using a higher temperature, lower pressure, a sweep gas, combinations thereof, etc.) to regenerate the sorbent bed for future use. The desorbed stream of ammonia can be vented to the atmosphere, destroyed using an ammonia decomposition reactor (e.g., a catalyst such as nickel, ruthenium, iron, cobalt, etc. coated on an electrically heated monolith operable to achieve a decomposition temperature between about 200 to 800° C. and/or supply the endothermic heat of reaction for ammonia decomposition into nitrogen and hydrogen) before venting, used as a dopant for a CVD process, used a chemical for other chemical reactions or processes, and/or can otherwise be handled.

Thus, certain embodiments of the invented process and apparatus result in high purity methane that can be compressed and recycled back to the CVD machine.

### 4.6 Example 5: Continuous Flow Results

Variants of the thermal reactor and/or method can achieve the results of continuous sorbent-enhanced reactor operation with upgraded methane production. Continuous flow operation (also “continuous operation” or “continuous mode”) can be understood as continuous flow of feedstream(s) into the thermal reactor and continuous flow of effluent/product stream(s) out from the reactor. In continuous operation, the flowrates into the reactor, and the flowrates of effluent from the reactor can vary, but said flowrates in and out of the reactor, once the reactor is started up (and at the preferred operating conditions so the reactor is at steady state flow), will each be greater than zero. Therefore, continuous operation is differentiated from batch operation wherein the batch steps can be described as: charging feedstock into the reactor; closing the reactor; adjusting conditions such as temperature, pressure, sorption chemical reactions taking place, reaction time, and/or other conditions; and opening the reactor for venting and/or drainage to obtain the products from said sorption and/or chemical reactions. The duration of the continuous flow reactor operation or “run” can last various lengths of time, for example at least 100 hours (e.g., 150 hrs, 200 hrs, 400 hrs, 500 hrs, 750 hrs, 1000 hrs, etc.). It should be understood, also, that certain embodiments of the operation can include reactor/process shut-down or “down-time” (e.g., for repairs, recharge of sorbent/catalyst, regeneration, low power availability, limited feed availability, low product demand, etc.). Thus, certain embodiments can be described as being “continuous” operations, where operation is stopped for periods of time for said maintenance, availability or demand reasons, or other reasons. In certain embodiments, such “discontinuous” operations can run with nonzero inlet and outlet flow rates in the range of 1 minute to 100 hours (e.g., 10 minutes, 30 min, 1 hr, 2 hrs, 5 hrs, 10 hrs, 20 hrs, 50 hrs, 75 hrs, etc.), between shut-downs to accommodate availability of intermittent power and/or can be inclusive of a process that cycles between two or more reactors with the use of valves.

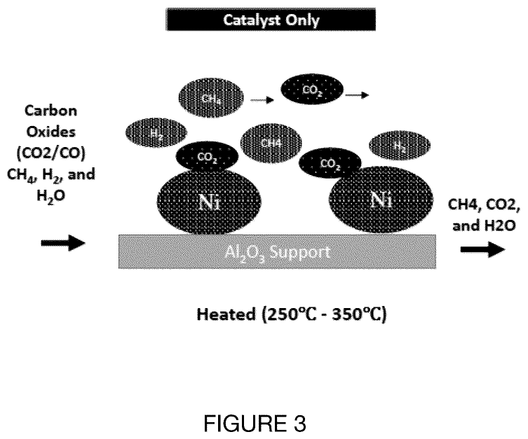

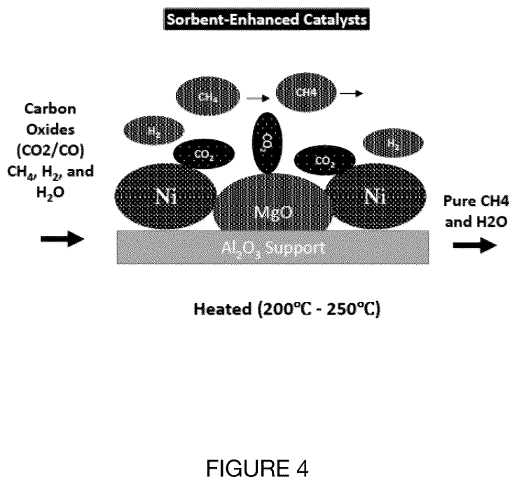

In certain embodiments of continuous operation, for example, by using a sorbent enhanced catalyst reactor system such as portrayed in FIG. 4 and the upper portion of FIG. 7, as opposed to a catalyst-only reactor system such as portrayed in FIG. 3 and the lower portion of FIG. 7, conversion of carbon oxides to methane close to 100% may be achieved. To achieve the same conversion with only catalyst (i.e., excluding sorbent), a methanation reactor would typically have to be much larger and/or operated under significantly higher pressure and/or temperature (recognizing that the reaction is equilibrium limited and only incomplete conversion is possible at elevated reaction temperature).

Although this disclosed technology has been described above with reference to particular means, materials and embodiments, it is to be understood that the disclosed technology is not limited to these disclosed particulars but extends instead to all equivalents within the broad scope of this disclosure including the following Example Claims and the drawings. For instance, while illustrative examples use specific numerical values within the broader range of possible waste stream compositions, variations of the technology can be shown to be effective across wider ranges of values (and the numerical values are merely used for illustrative purposes and are not intended to limit the inventive concept to the illustrative example compositions).

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the preceding system and/or method can be used with, in addition to, in lieu of, or otherwise integrated with all or a portion of the systems and/or methods disclosed in the applications mentioned above, each of which are incorporated in their entirety by this reference.

As used herein, “substantially” or other words of approximation (e.g., “about,” “approximately,” etc.) can be within a predetermined error threshold or tolerance of a metric, component, or other reference (e.g., within 0.001%, 0.01%, 0.1%, 1%, 5%, 10%, 20%, 30% of a reference), or be otherwise interpreted.

As a person skilled in the art will recognize from the previous detailed description and from the figures and claims, modifications and changes can be made to the preferred embodiments of the invention without departing from the scope of this invention defined in the following claims.

### 6. Specific Examples

A numbered list of specific examples of the technology described herein are provided below. A person of skill in the art will recognize that the scope of the technology is not limited to and/or by these specific examples.

1A. A process for upgrading methane-laden waste streams, comprising: introducing a waste stream comprising methane, hydrogen, and carbon and nitrogen contaminants into a core reactor containing a catalyst enhanced by sorbents and promoters; retaining carbon dioxide near the active catalyst site via the sorbents enhanced phase, away from the bulk flow path; converting carbon contaminants to methane at a rate greater than 90% or greater than about 90%, and operating the core reactor at a pressure range of 0.5 to 2 atmospheres about 0.5 to 2 atmospheres.

1B. A process for CO₂ conversion, comprising: a material comprising an active catalyst and adsorbent coated on reacting chamber walls circumscribing at least 40% of the process gas flow path, wherein, the gas flow path is substantially open where the gas flow comprising CO₂ and hydrogen reacts to form methane within the catalyst and adsorbent coating, wherein, the conversion of CO₂ is greater than 60% per pass.

Variations of 1A or 1B with one or more of: catalyst and adsorbent at least 50%, at least 60%, greater than 90%, greater than 95% of wall circumscribing the process flow channel; catalyst and adsorbent coating greater than 0.7 mg/cm²; catalyst and adsorbent ratio from 1:100 to 1:20; conversion greater than 70%, greater than 80%, greater than 90%, greater than 95%, greater than 99% per pass; catalyst comprises at least one of the following Ru, Fe, Ni, K, Co, Mn, Rh, or Pt; adsorbent comprises at least one of the following Li, Na, K, Rb, Cs, Be, Mg, Ca, Sr, Ba, or oxides thereof (e.g., Na₂O, MgO, CaO, Li₂O, SrO, BaO, K₂O, Rb₂O, Cs₂O, BeO, etc.); catalyst and adsorbent are mixed in a slurry prior to coating; catalyst and adsorbent are coated in distinct layers, wherein the catalyst is coated closest to the wall that defines the flow channel; or catalyst is coated farthest from the wall that defines the flow channel; the process flow is laminar in the reaction chamber; the thickness of the combined catalyst and adsorbent layer or layers is at least 5 micron and less than 500 microns (e.g., 10 to 200 microns, 20 to 100 microns, etc.), the process operates in a continuous manner with a run time of at least 100 hours; the process operates in a “discontinuous” manner with a run time between 1 minute and 100 hours (e.g., 10 minutes and 10 hours); or the process operates without active cooling and heat dissipates by heat losses; the process operates with active cooling where internal heat transfer channels are located along at least 5% of the process reaction chamber (e.g., about 5 to 100% of the process channel, 10 to 50% of the process channel, etc.); the process operates with two or more stages in series, where an intervening heat exchanger removes heat generated in the first stage before process gas flows to at least a second stage reactor; a process with a pressure drop less than 150000 Pa or less than about 150000 Pa (e.g., 100000 Pa, 50,000 Pa, 10,000 Pa, 1,000 Pa, etc.) during the production of methane.

2. The process of specific example 1A or 1B, wherein the conversion rate of carbon contaminants to methane is more than 95% or more than about 95%.

3. The process of specific example 1A or 1B, wherein the conversion rate of carbon contaminants to methane is more than 99% or more than about 99%.

4. A system for converting waste streams into high-purity methane, the system comprising: a core reactor designed to operate at near-atmospheric pressure, embedded with catalysts coated on a substrate with defined flow paths; sorbents and promoters within the core reactor that increase catalysis rate and the retention of carbon dioxide near the catalyst sites, and supplemental processes upstream and downstream of the core reactor for purifying out trace contaminants.

5. The system of specific example 4, where the core reactor achieves a conversion rate of greater than 90% in a single pass.

6. The system of specific example 4, where the core reactor achieves a conversion rate of greater than 95% in a single pass.

7. The system of specific example 4, where the core reactor achieves a conversion rate of greater than 99% in a single pass.

8. A method for producing high-purity methane suitable for transportation in natural gas infrastructure, the method comprising: treating a stream containing, comprising, consisting essentially of, or consisting of methane, carbon dioxide, and contaminants using a core reactor with sorbent-enhanced catalysts, operating the core reactor at low temperatures around 200° C., and achieving conversion with near 100% selectivity towards methane.

9. The method of specific example 8, wherein the selectivity towards methane is greater than 95%.

10. The method of specific example 8, wherein the selectivity towards methane is greater than 99%.

11. The method of specific example 8, wherein the selectivity towards methane is greater than 99.9%.

12. Use of the process according to any one of specific examples 1A, 1B, or 2-11 for converting a primarily hydrogen-containing stream with methane and carbon impurities into ultra-high purity methane for use in industrial applications, including chemical vapor deposition, atomic layer deposition, and semiconductor manufacturing.

We claim:

1. A method comprising:
a) receiving a waste stream from chemical vapor deposition growth of a carbonaceous material, the waste stream comprising hydrogen, methane, carbon oxides, non-methane hydrocarbons, and nitrogen species;
b) hydrogenating the waste stream to reduce unsaturated non-methane hydrocarbons or unsaturated nitrogen species to saturated non-methane hydrocarbons or saturated nitrogen species;
c) pre-reforming the saturated non-methane hydrocarbons or saturated nitrogen species to the carbon oxides, water, and nitrogen;
d) methanating the carbon oxides to upcycled methane;
e) producing ammonia from the nitrogen; and
f) using the upcycled methane for further chemical vapor deposition growth of the carbonaceous material.

2. The method of claim 1, wherein steps b) through e) are each performed in an electrified thermal reactor.

3. The method of claim 2, wherein the electrified thermal reactor comprises a bifunctional catalyst comprising a catalyst and a sorbent.

4. The method of claim 2, wherein a separate electrified thermal reactor is used for each of steps b) through e).

5. The method of claim 4, wherein the electrified thermal reactor for performing step b) comprises a catalyst comprising at least one of nickel, platinum, palladium, ruthenium, or rhodium; wherein the catalyst is supported on at least one of silica, alumina, ceria, silicon carbide, or titania.

6. The method of claim 5, wherein the catalyst is associated with a promoter comprising molybdenum.

7. The method of claim 4, wherein the electrified thermal reactor for performing step c) comprises a catalyst comprising rhodium; wherein the catalyst is supported on at least one of silica, alumina, ceria, silicon carbide, or titania.

8. The method of claim 4, wherein the electrified thermal reactor for performing step d) comprises a catalyst comprising ruthenium, rhodium, or nickel; wherein the catalyst is supported on at least one of silica, alumina, ceria, silicon carbide, or titania.

9. The method of claim 4, wherein the electrified thermal reactor for performing step e) comprises a catalyst comprising ruthenium or iron; wherein the catalyst is supported on at least one of silica, alumina, ceria, silicon carbide, or titania; wherein the catalyst is associated with a promoter comprising at least one of cesium or barium.

10. A method comprising:
receiving a waste stream from chemical vapor deposition growth of a carbonaceous material, the waste stream comprising hydrogen, methane, and carbon oxide;
treating the waste stream using a thermal reactor comprising sorbent-enhanced catalysts to convert the carbon oxide into upcycled methane;
wherein the upcycled methane comprises is used for further chemical vapor deposition growth of the carbonaceous material.

11. The method of claim 10, wherein the thermal reactor is operated at a temperature between 100° C. and 300° C.

12. The method of claim 11, wherein when reactions performed during treating the waste stream are net exothermic, the thermal reactor is operated intermittently to prevent a temperature from exceeding about 300° C.

13. The method of claim 10, wherein prior to using the upcycled methane for further chemical vapor deposition growth of the carbonaceous material, hydrogen is separated from the upcycled methane by using a hydrogen fuel cell to oxidize the hydrogen or using a hydrogen selective membrane to filter the hydrogen from the upcycled methane.

14. The method of claim 10, further comprising purging the upcycled methane to remove inert contaminants.

15. The method of claim 10, further comprising sorbing contaminants out of the upcycled methane.

16. The method of claim 10, wherein the waste stream further comprises non-methane hydrocarbons, wherein treating the waste stream comprises at least one of hydrogenation of unsaturated hydrocarbons, prereforming of the non-methane hydrocarbons into the carbon oxides and water, and methanation of the carbon oxides.

17. The method of claim 16, wherein the waste stream further comprises nitrogen or reactive nitrogen species, wherein treating the waste stream further comprises producing ammonia from the nitrogen or reactive nitrogen species.

18. The method of claim 16, wherein the thermal reactor comprises a plurality of thermal reaction modules, wherein each thermal reaction module of the plurality of thermal reaction modules is operated at a different temperature, with a different catalyst, with a different support, or with a different sorbent from the other thermal reaction modules.

19. The method of claim 10, wherein the waste stream comprises about 75% and 99.99% hydrogen by mass, 0.01% and 25% methane by mass, 0.0001% and 10% carbon monoxide by mass, and 0.0001% and 10% carbon dioxide by mass; wherein the total percentages add up to 100%.

20. The method of claim 18, wherein the waste stream further comprises between 0.1 ppm and 5000 ppm non-methane hydrocarbons, 0.01 ppm and 5000 ppm inert gases, and 0.01 ppm and 200 ppm reactive nitrogen species.
